(19) United States
(12) Patent Application Publication
(10) Pub. No.: US 2025/0065063 A1
(43) Pub. Date: Feb. 27, 2025

(54) VENTILATION ASSESSMENT SYSTEM, FACE MASK, AND METHOD

(71) Applicant: The Regents of the University of Michigan, Ann Arbor, MI (US)

(72) Inventors: Jacqueline HANNAN, Poughkeepsie, NY (US); Gary WEINER, Ann Arbor, MI (US); Leia STIRLING, Ann Arbor, MI (US)

(21) Appl. No.: 18/813,171

(22) Filed: Aug. 23, 2024

Related U.S. Application Data
(60) Provisional application No. 63/534,499, filed on Aug. 24, 2023.

(51) Int. Cl. A61M 16/00; A61M 16/06

(52) U.S. Cl. CPC A61M 16/0003; A61M 16/0605; A61M 2016/0027; A61M 2205/15; A61M 2205/332; A61M 2205/502; A61M 2240/00

(57) ABSTRACT

A method of assessing ventilation and a system for assessing ventilation integrate sensor-based force outputs with graphical, real-time feedback for positive pressure ventilation (PPV), particularly for infants. In one implementation, the method includes receiving a measurement of an applied force between a face mask and a face of a patient from a first sensor located at least partially between the face mask and the face of the patient, receiving a measurement of an overall force applied to the patient from a second sensor, and contextualizing the measured applied force and the measured overall force to provide a graphical user interface.

### TECHNICAL FIELD

The present disclosure relates generally to assessment systems and methods for administering positive pressure ventilation (PPV), particularly in neonatal applications.

### BACKGROUND

Positive pressure ventilation (PPV) is a form of respiratory support that delivers pressurized air from an external source to the lungs through an interface applied to the mouth and nose (face mask) or directly into the trachea (endotracheal tube). PPV can be the single most effective step in the resuscitation of compromised newborn infants. It is used when newborns are not breathing, if they are gasping for air, or if their heart rate is below 100 beats per minute within the first few minutes after birth. Each year, approximately 5-10% of newborns will receive PPV with a face mask. Care must be taken when holding the face mask during PPV, as applying too much pressure has potential to injure the infant, while applying too little pressure will contribute to leakage and insufficient air delivery. Currently, no previous research has attempted to quantify the amount of force applied to the infant at key points of contact between the face mask and the infant's face. As a result, there are no recommendations for applied forces during neonatal PPV at this time. Additionally, there is no standardized tool to measure and communicate the interaction pressures occurring during ventilation, which would be useful for optimizing the amount of force needed to properly apply a face mask and achieve effective neonatal PPV. Such a tool could support clinical practice and assist with training in PPV.

During ventilation, achieving an adequate seal of the mask to the face helps to prevent air from escaping out the sides of the mask and to ensure adequate air delivery. Although the amount of pressure applied at key locations on an infant's face had not previously been quantified, previous studies have shown that between 24 to 59 percent of the air flow leaving the PPV device leaks around the face mask. This leak can interfere with the effectiveness of PPV, resulting in delayed recovery of the newborn and initiation of more invasive procedures. Additionally, prior work has shown that the majority of leakage occurs during the inflation phase of the breath rather than deflation, indicating that leakage primarily removes air that should have been delivered to the infant's lungs. The technique used for holding the face mask flush with the infant's face may affect leakage. Prior research has identified multiple techniques that are often applied when administering PPV, including using either one or two hands to hold the mask and adjusting the position of the hand applying pressure in different locations (e.g., evenly distributed around mask rim, pressure at chin and nose, etc.). Since no quantitative tool for measuring the applied forces previously existed, it is unclear how the varying techniques of holding the mask while administering PPV affect the range of interaction pressures applied to the infant's face and the amount of air flow delivered by the PPV device.

### SUMMARY

In accordance with one embodiment, there is provided a method of assessing ventilation, comprising the steps of: receiving a measurement of an applied force between a face mask and a face of a patient from a first sensor located at least partially between the face mask and the face of the patient, receiving a measurement of an overall force applied to the patient from a second sensor; and contextualizing the measured applied force and the measured overall force to provide a graphical user interface.

In some embodiments, the graphical user interface includes a time series plot of an output from each of the first sensor and the second sensor. The method may further comprise the step of accounting for an initial unloaded value of each of the first sensor and the second sensor in the time series plot. The measurement from the first sensor, the measurement from the second sensor, or the measurements from both of the first sensor and the second sensor can be used to provide an indication of a potential airway obstruction. The first sensor can be one of a plurality of face mask sensors. The method may also include the steps of comparing each sensor measurement of the plurality of face mask sensors to assess similarity of the applied force across the face mask. An increased relative pressure at a zygomatic arch sensor, or any one of the sensor locations, in the plurality of face mask sensors can be used to provide an indication of a potential injury. In some embodiments, the second sensor is an under-head sensor configured to be located on an opposite side of a head of the patient from the face. The method may also comprise the step of determining a relationship of the applied force and/or the overall force to one or more of a mask leak measurement, a peak inflation pressure, and a tidal volume. The analysis can be adjusted depending on a one-hand usage pattern or a two-hand usage pattern, and may include calculating a force asymmetry index. A non-transitory, computer-readable storage medium can store instructions thereon that when executed by one or more electronic processors causes the one or more electronic processors to carry out the method.

In accordance with another embodiment, there is provided a ventilation assessment system comprising a plurality of face mask sensors configured to be situated between a face mask and a face of a patient to measure an applied force between the face mask and the face of the patient, and an under-head sensor configured to measure an overall force applied to the patient.

In some embodiments, the plurality of face mask sensors is attached to the face mask or integrated into the face mask. The patient may be an infant manikin and the plurality of face mask sensors can be integrated into or onto the face of the infant manikin. The under-head can be integrated into or onto a head of the infant manikin. The under-head sensor can be integrated onto the head of the infant manikin with a strap. The under-head sensor can be separate from the head of the infant manikin and placed on the surface underneath the head of the infant manikin. In some embodiments, the plurality of face mask sensors is integrated with a conformational interlayer. The conformational interlayer can include two or more thermoplastic layers that have a face contour profile, and the plurality of sensors are sandwiched between the two or more thermoplastic layers.

In accordance with another embodiment, there is provided a face mask for a ventilation assessment system comprising a nasal bridge sensor configured to measure an applied force across a nasal bridge of a patient, and a mentum sensor configured to measure an applied force at a mentum of the patient. The nasal bridge sensor and the mentum sensor are at least partially aligned with and at least partially overlapping a longitudinal axis of the face mask. The mask may include a right zygomatic arch sensor configured to measure an applied force at a right zygomatic arch of the patient and a left zygomatic arch sensor configured to measure an applied force at a left zygomatic arch of the patient.

It is contemplated than any of the above-listed features can be combined with any other feature or features of the above-described embodiments or the features described below and/or depicted in the drawings, except where there is an incompatibility of features.

### BRIEF DESCRIPTION OF THE DRAWINGS

Preferred example embodiments will hereinafter be described in conjunction with the appended drawings, wherein like designations denote like elements, and wherein:

FIG. 1 is a partially schematic view of one embodiment of a ventilation assessment system;

FIG. 2 illustrates part of a ventilation assessment system according to one embodiment;

FIG. 3 shows example sensor locations for the ventilation assessment system of FIGS. 1 and 2;

FIG. 4 shows force readings at each of the sensor locations of FIG. 3 for the desired under-head forces with the standard deviation shaded;

FIG. 5 is a box plot of proximal air flow measurement at different increments of under-head pressure;

FIG. 6 is a box plot of mean force at various sensor locations, such as the locations shown in FIG. 3;

FIG. 7 is a box plot of mean under head force vs. mask type;

FIG. 8 is a box plot of mean under head force vs. device type;

FIG. 9 is an exploded view of another embodiment of a ventilation assessment system;

FIG. 10 shows a sensor adapter from the system of FIG. 9;

FIG. 11 shows a bottom view of the sensor adapter from the system of FIG. 9;

FIG. 12 shows another embodiment of a ventilation assessment system;

FIG. 13 shows a sensor adapter that can be used with the ventilation assessment system of FIG. 12, for example;

FIG. 14 shows a sensor adapter that can be used with the ventilation assessment system of FIG. 12, for example;

FIG. 15 shows a glove that can be used with a ventilation assessment system;

FIG. 16 shows another embodiment of a ventilation assessment system;

FIG. 17 shows a sensor adapter or mask that can be used with the ventilation system of FIG. 16;

FIG. 18 is a partial cross-section view taken through line 18-18 of FIG. 17; and

FIG. 19 shows an example circuit schematic that can be used with a ventilation assessment system.

### DESCRIPTION OF THE ILLUSTRATED EMBODIMENTS

Described herein is a ventilation assessment system and method that can be implemented as an integrated hardware and software system for characterizing interaction pressures during neonatal positive pressure ventilation (PPV). PPV can provide life-saving support to newborns via face mask and air flow source. PPV requires a careful balance of applied force on the face mask to provide enough pressure to form a tight seal for air flow without delivering too much pressure that could injure the infant. Previously, there was no tool or quantitative technology to assist in training clinical staff on applying the proper amount of pressure to achieve effective ventilation. The present disclosure describes a sensor system that can be used to monitor forces and pressures at key locations on a newborn's head and the proximal air flow rate between the ventilator and the face mask. The present systems can provide a clinician with guidance as to minimum pressures that can be used to form a complete seal of the face mask using contextualized real-time feedback. Additionally, terminal feedback from the system may be gathered and used, whether to modify system parameters, adjust testing or assessment criteria, or for any other operable purpose.

Providing a tool to support clinical feedback and technique examination while administering PPV requires an interface that supports data interpretation. Contextualization and visual format are two key contributors to quickly inform the user of a situation. Visual displays can provide important feedback to the user about the current system state, allowing the user to monitor key metrics while performing a task. Providing data as a time series gives the user the ability to easily assess changes of the system state. In the case of synchronous feedback, contextualizing the range of the datapoints helps provide useful insights about the acceptable values of a variable. The contextualization becomes particularly beneficial when an operator needs to perform multiple tasks or monitor multiple sources of information, requiring effective divided attention. A common means of failure for divided attention is when the operator becomes too fixated on one information source, leading to insufficient monitoring of other tasks and information. An operator may not understand the necessary information if the presented data is unclear or overly complex. Presenting PPV operators with metrics lacking context may become overwhelming and unclear. Visual thresholds allow for quick analysis of acceptable values, which can be advantageous when performing a time-sensitive task such as neonatal ventilation. Therefore, there is a need to devise a minimum amount of pressure required to form a complete seal of the face mask, aiding in the creation of a lower threshold for the visual time series data. To support the tracking of interaction pressures and flow rate during PPV, the interactive sensor tool described herein can be used to convey essential information to the individual performing PPV. This system can communicate interaction pressures between the ventilation device and the newborn at key locations on the face and under the head, in addition to monitoring proximal air flow rates between the ventilator and the newborn. The sensor system displays these real-time force and air flow metrics on a graphical user interface (GUI) in the form of a time series. For the purposes of this system, a manikin representing a full-term newborn infant was used in conjunction with the sensors. The goals of the sensor system are to serve as a training tool to ensure PPV is delivered in a safe and effective manner, and to serve as a research tool to investigate future hypotheses regarding neonatal ventilation methods. In other embodiments, however, the teachings herein may be applicable to actual patient treatment efforts, as opposed to just being a training aid for clinicians.

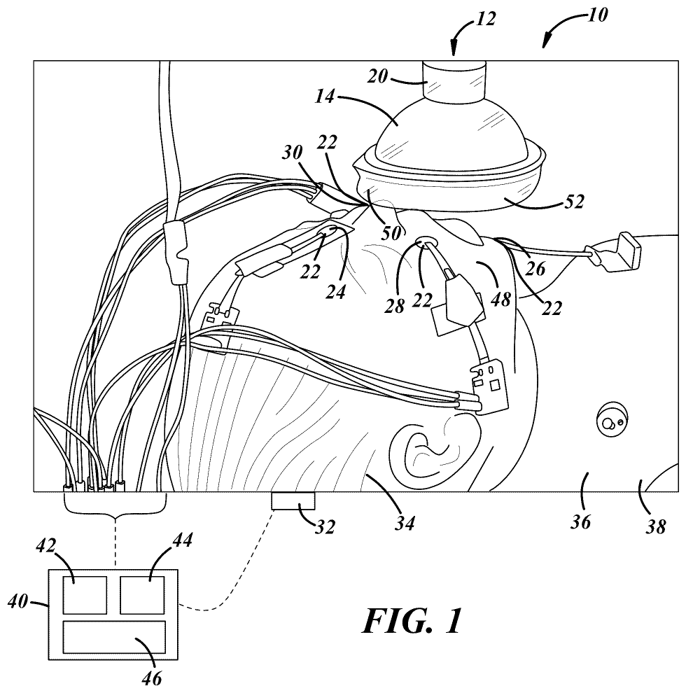

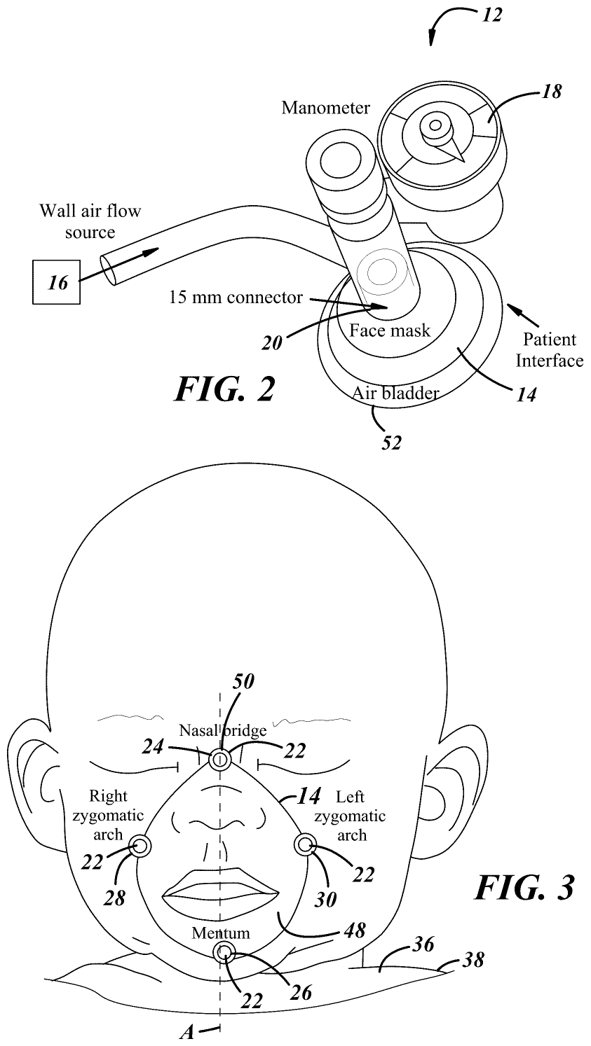

FIG. 1 schematically illustrates a ventilation assessment system 10. The ventilation assessment system 10 includes a PPV device 12 which has a face mask 14. FIG. 2 shows an example PPV device 12; however, the system may use or incorporate a number of different styles of PPV devices, all of which include an air source 16, manometer (pressure gauge) 18, and standard connection 20 to the patient interface or face mask 14. As shown in FIG. 1, the ventilation assessment system 10 includes a plurality of face mask sensors 22, including in this embodiment, a nasal bridge sensor 24, a mentum sensor 26, a right zygomatic arch sensor 28, and a left zygomatic arch sensor 30. An under-head sensor 32 is also schematically illustrated, and is situated behind the head 34 of the patient 36. The patient 36 may be an actual live person (adult or neonate), a manikin, or in an advantageous embodiment as illustrated, an infant manikin 38.

The sensors 22, 32 are coupled to a computer 40 having an electronic processor 42 and memory 44. A graphical user interface 46 is also provided to show a user real-time, contextualized feedback from the sensors 22, 32. Additionally, information gathered previously (terminal feedback) may be used to modify the graphical user interface 46, such as to provide ideal examples for applied force, to cite one example. The sensors 22, 32 may be directly coupled via a wired connection as shown, or any other operable coupling, such as Wi-Fi, short-range wireless communication (SRWC) or the like. The processor 42 may be any type of device capable of processing electronic instructions, including microprocessors, microcontrollers, host processors, controllers, and application specific integrated circuits (ASICs). It can be a dedicated processor used only for the computer 40, or it may be shared or used with other diagnostic or assessment related devices. Processor 42 executes various types of digitally-stored instructions, such as software or firmware programs stored in memory 44. For example, processor 42 can execute programs or process data to carry out at least part of the assessment methods discussed herein. Memory 44 may be a temporary powered memory, and any non-transitory computer readable medium, or other type of memory. For example, the memory can be any of a number of different types of RAM (random-access memory), ROM (read-only memory), solid-state drives (SSDs), hard disk drives (HDDs), magnetic or optical disc drives, etc. The processing of data from sensors 22, 32 may be accomplished by a dedicated computer 40 as schematically illustrated, or on an existing system or distributed architecture, to cite a few examples. As detailed herein, in one implementation, the sensors 22, 32 and the flow meter 18 were connected to an ARDUINO UNO as the processor 42 using I2C communication for data collection.

To quantify and monitor the forces applied to the manikin's face 48 and head 34, five SINGLETACT (PPS UK Limited, Glasgow, UK) pre-calibrated capacitive force sensors 22, 32 with a maximum load of 100 N (22 lb) were used. The sensors 22, 32 in the illustrated embodiments have a diameter of 8 mm and a thickness of 0.30 mm. The small profile (e.g., thickness less than or equal to 0.5 mm) helps to ensure that the thickness of the sensor will not interfere with the simulated ventilation. Four of the sensors 22 were placed at key pressure points on the face 48: nasal bridge sensor 24, mentum sensor 26, and left and right zygomatic arch sensors 28, 30 (referenced from the manikin). The fifth sensor 32 was placed directly under the manikin's head 34, where it makes contact with the table surface. In some embodiments, a digital scale or another sensor type may be used to measure the overall force under the head. These locations were selected to measure pressure at points of contact with bone on the face and support spacing around the face mask 14, as well as to measure the amount of force applied to the overall head 34. To measure the proximal air flow through the ventilator and the face mask 14, the SENSIRION SFM3000 digital flow meter was used. This flow meter captures bi-directional flow, displaying the net flow of air within the flow channel.

It is possible to use other types of sensors and other configurations for the sensors 22, 32. For example, instead of capacitive force sensors, other sensor types are possible, such as force sensitive resistors, smart materials, microfluidic capillaries with conductive fluid, etc. Additionally, the spacing of the sensors 22 around the mask 14 may vary; however, the illustrated locations are particularly beneficial given their respective proximity to bones in the face 48 of the infant 36. The spacing of the sensors 22 around the mask 14 provide a symmetrical arrangement with respect to a longitudinal axis A of the mask. In this embodiment, the nasal bridge sensor 24 and the mentum sensor 26 are aligned with and overlap the longitudinal axis A of the face mask 14. This arrangement can be particularly beneficial with either a round or an anatomically shaped mask (with the anatomically shaped mask shown in FIG. 3 having an apex 50 at the nasal bridge sensor 24). Having the position of the nasal bridge sensor 24 and the mentum sensor 26 aligned with the longitudinal axis A may provide a more accurate comparison of applied pressure at the mentum sensor 26 as opposed to other locations along the face mask 14, which may then be used to determine a potential airway obstruction. The sensors 22, 32 may be integrated with the infant manikin 38, on an inflatable rim 52 of the mask 14, or be separate and then applied between the patient 36 and the mask 14 (e.g., as a custom interface that attaches to the mask 14).

In the illustrated implementation, the PPV device 12 that was used was the NEO-TEE hand operated T-piece ventilation device and an anatomic face mask 14 with an inflatable rim 52. The connector 20 components of the T-piece ventilator were adapted to form a tight seal with both ends of the flow meter 18. These connectors 20 were reinforced with tape to help ensure no air leakage occurred. In conjunction with the sensors 22, 32, a LAERDAL full term newborn manikin 38 was used. Other manikin 38 types are certainly possible, including the SimNewB, Newborn Anne, Premature Anne, and the Neonatal

Intubation Trainer, to cite a few examples. These manikin types can be quite sophisticated, but in at least some implementations, a more simple manikin can be used, which decreases the cost of the system 10. The system 10 was and can be specifically designed for neonate applications, where the face mask 14 is proportionally smaller than a standard adult size mask (e.g., about 35-45 mm in diameter measured between the left and right sensors 28, 30, compared with an adult medium size mask which is about 100 mm in diameter, and thus the infant size is typically less than half the diameter of the adult size). The smaller size of the patient 36 and the mask 14 can result in more challenges to a user trying to ventilate the infant, as control of pressure can be a greater concern. Additionally, the newborn manikin 38 has an anatomically correct or structured airway, as oppose to a simple tube, which can be better at assessing potential obstruction and enhance the training experience.

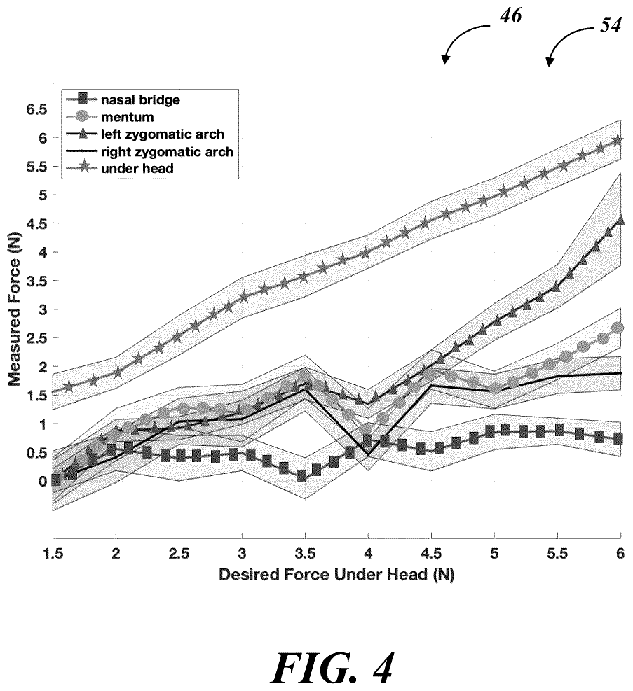

With reference to FIG. 4, in addition to the physical hardware components, a GUI 46 was created to provide a real-time graph 54 of the time series data, allowing for observation of the data during use of the system 10. The GUI 46 was also used for recording data to a .csv file for analysis of the interaction pressures and air flow rate. In this embodiment, a two-hand C-E technique was used to hold the face mask 14 to the manikin 38, and it is possible to use the hand positioning as an input to the system and methods herein, and adjust the force thresholds for variations in positioning. Air flow and pressures applied to the manikin's face and head were measured at increments of applied under-head force throughout the trial. To help develop force thresholds, prior to the start of data collection, a pressurized wall air source was set to 10 L/min, which was confirmed by measuring the flow using the proximal flow meter. In some embodiments, the thresholds may be displayed on the GUI 46 or used as input for the GUI. To begin the data collection, the GUI 46 was initiated and the ventilator 12 was placed on the manikin's face 48 without applying any pressure, just allowing it to rest in position. Thirty seconds of data were collected in this position, and the baseline pressure under the head from sensor 32 was observed. After noting the baseline under-head force reading, incremental pressure was applied to the face mask 14 in a uniform method, with the goal of adding 0.5 N of under-head pressure for 30 seconds at a time. The operator observed the under-head pressure time series data and attempted to keep the value as close to the goal value as possible for the entire duration of the 30 seconds. After 30 seconds, the goal was to increase another 0.5 N, once again attempting to stay at the given value, and so on. This incremental increase in applied pressure was performed until a max value of 6.0 N. Using this time synchronized information, the minimum interaction pressures at the sensors 22 were estimated that were required to form a complete seal of the ventilator face mask 14 to the manikin face 48.

To set thresholds for applied force for each of the sensors 22, 32, the data can refined in certain ways to help provide a more accurate output for the GUI 46. For example, during analysis of the data, the middle twenty seconds of each thirty second increment can be extracted for further analysis. The initial un-loaded values of each force sensor 22, 32 at a resting state on the manikin 38 were subtracted from the time series such that all sensors 22, 32 had a zero datum when unloaded. By subtracting the resting state values of the force sensors 22, 32, any small changes in conductance due to bending of the sensors on the uneven surface of the manikin's face 48 can be accounted for. The mean and standard deviation were calculated for all five force sensors 22, 32 and the flow meter 18 at each increment of the desired pressure under the head 34.

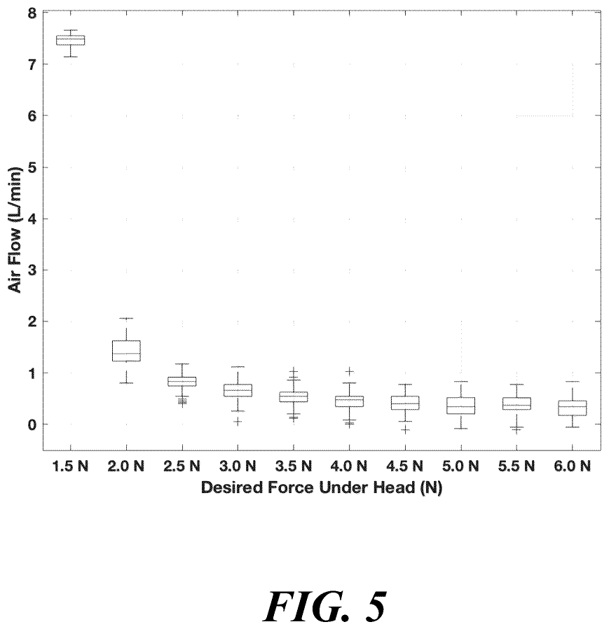

With reference to FIG. 5, independent t-tests were used to examine differences in air flow rate between under-head force conditions. The initial air flow rate when the mask 14 was placed on the manikin's face 48 without any pressure applied was approximately 7.45 L/min, representing approximately 75% leakage of the total air flow. The under-head resting force was approximately 1.5 N. The air flow decreased non-linearly, with smaller decrements as the applied force increased under the head 34. The leakage rates for 3.5 N, 4.5 N, and 5.5 N were approximately 5.3%, 4.1%, and 3.7% respectively. Independent t-tests support that there was no significant difference between the air flow rate at 4.0 N and any of the higher values (at 4.5 N: t(198.16)=0.5125, p=0.6089, at 5.0 N: t(190.11)=1.059, p=0.2909, at 5.5 N: t(179.99)=0.8255, p=0.4102, at 6.0 N: t(167.75)=1.266, p=0.2073).

Upon determining that 4.0 N of reference under-head force is the value at which the air flow rate does not significantly differ for the given reference under-head sensor, the behavior of pressures at other locations on the manikin 38 throughout the trial were analyzed. The mean force measured under the head 34 was 4.004 N, with observed forces of 0.720 N at the nasal bridge sensor 24, 0.887 N at the mentum sensor 26, and 0.458 N and 1.344 N at the right and left zygomatic arch sensors 28, 30, respectively. There are features in FIG. 3 that provide details on how the applied force was distributed across the manikin face 48 for each given under head force magnitude. At 3.5 N of force measured under the head 34, the force measured at the nasal bridge drops and the forces at the three other locations increase. At 4.0 N of force, the measured values increased at the nasal bridge and dropped at all other face locations.

Differentiation in the pattern of forces when approaching larger increment values was also observed. Towards the end of the trial, the forces observed at the left zygomatic arch sensor 30 increased in response to the positioning of the applied force. This comparative increase in the applied force at the left zygomatic arch sensor 30 may be accounted for in the applied force thresholds used for the GUI 46 (e.g., a warning may not be provided to a user even though the applied force at the left zygomatic arch sensor 30 is comparatively higher). In an advantageous embodiment, the minimum amount of force required to form a complete seal between the mask 14 and the newborn manikin face 48 is used as one or more thresholds (e.g., applied forces less than 0.5 N at the mentum sensor 26 could trigger a warning whereas forces less than 1.0 N at the left zygomatic arch sensor 30 trigger a warning). Thresholds may also include upper limits that could indicate a potential injury. For example, too much applied pressure indicated by one of the zygomatic arch sensors 28, 30 could cause injury to an infant's eye or undesirably slow the infant's heartrate.

The force measured under the head by the under-head sensor 32 is not directly equal to the force applied to the mask 14 as sensed by the sensors 22, as the face mask 14 and manikin 38 have some deformation from the applied force. As the force magnitude measured under the head 34 increased, the air flow rate decreased non-linearly. The resulting forces on the manikin's face 48 were lower than expected. Interestingly, the values recorded from the force sensors 22 on the face did not appear to increase linearly with the pressure measured under the head for all sensors. Instead, the forces increased at a slower rate, with some sensors locations reaching a plateau (e.g., at the nasal bridge sensor 24). This finding was in contrast to the expected result of observing increased applied force values at each location on the face 48 as the overall force applied to the head 34 increased. The plateau in may have occurred due to the available volume and air density within the air bladder on the rim 52 of the face mask 14 in combination with the material properties of the bladder. The applied forces generate an increased pressure within the bladder, which could have increased tension in the bladder material and expanded the contact surface area, leading to the forces distributed over a greater region that was not directly measured. Accordingly, the mask materials may be used as an input to the system 10 to adjust the GUI 46. The mask materials may be used to quantify the changes in surface area based on the bladder designs available and how that may affect interaction forces. Additionally, the nasal bridge is a less pronounced feature on the infant manikin 38, which aligns with the lower values observed at this location.

When one applies the pressure to the mask 14 uniformly, the values at specific points on the face 48 remain lower and were observed to plateau. However, if the general location of the applied force shifts, it is possible to produce a greater magnitude force at specific localizations on the face 48. In the trial, the left zygomatic arch sensor 30 was directly in front of the operator. The operator shifted the force application slightly forward rather than administering the force straight down, which generated the higher applied force at this location. A similar behavior is observed at 3.5 N of force under the head, where the force applied at the nasal bridge decreased and the force applied at the mentum increased during this period. These observations indicate that the operator shifted the force application slightly away from the nasal bridge and towards the mentum, as these two force locations counterbalance each other. Accordingly, it is possible for the system 10 to detect directional changes in applied force on the manikin face 48, which can be added as a contextual detail for the GUI 46.

Based on the force sensor outcomes at 4.0 N of under-head force via under-head sensor 32, an initial lower bound of 0.5 N for the face sensors 22 can be used, as data shows that consistent applied force should result in a minimum of 0.5 N at each face sensor location. Setting this threshold would support indication of a complete seal, as we observed approximately 5% air leakage at an under-head value of 4.0 N. This amount of leakage is much lower than measurements presented in previous studies (24% to 59%). The 0.5 N lower bound for the face sensors 22 will also support awareness of providing equal distribution of force across the face mask. As described, however, having variable thresholds for each sensor 22, along with changing said thresholds depending on the mask materials, hand positioning, etc. can be advantageous. Additionally, it may be helpful to predict and note with the GUI 46 when the location of pressure application shifts, to help support clinical staff in using proper form.

It is important to note that the results of these initial force measurements on the face 48 may not translate directly from the manikin 38 to an infant, and adjustments can be made to the system 10 depending on the desired implementation (e.g., training vs. treatment). The sensor system 10 can serve as a new tool for future hypothesis-based research questions, in addition to providing improved user feedback for training individuals in PPV. In some embodiments, the magnitude of applied force is compared to the magnitude of force measured under the head 34. This comparison could better inform clinical staff about the direct relationship between applied pressure and the amount of pressure received by the newborn 36 versus dissipated through the face mask 14. In addition to adding the measurement of applied pressure, the current electronic selections could be revised to best fit the context of neonatal ventilation. For example, sensors with a smaller force range could provide more sensitive feedback. Additionally, using a force sensor with a larger area under the head could capture all of the interaction points between the manikin's head and the work surface when used as the under-head sensor 32. Future hypothesis testing could use the system 10 to compare multiple variables within neonatal ventilation and provide insightful feedback to clinicians. These studies will help progress our understanding of neonatal ventilation from the perspectives of both advancing research and improving clinical implementation.

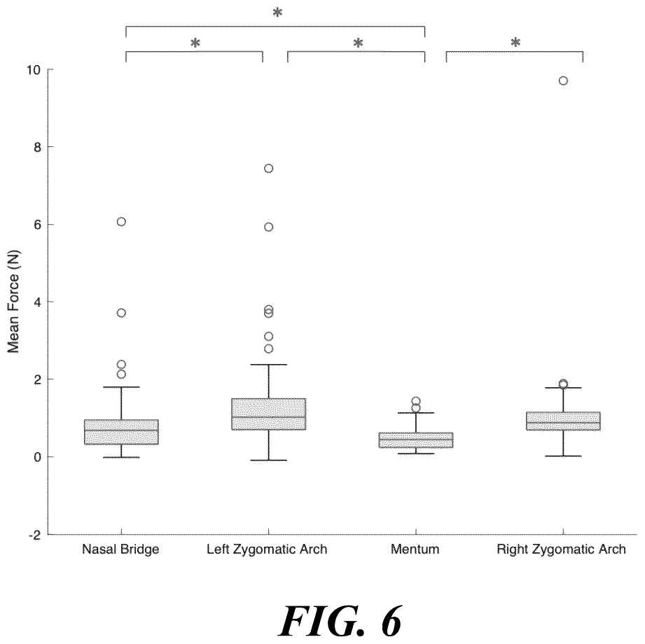

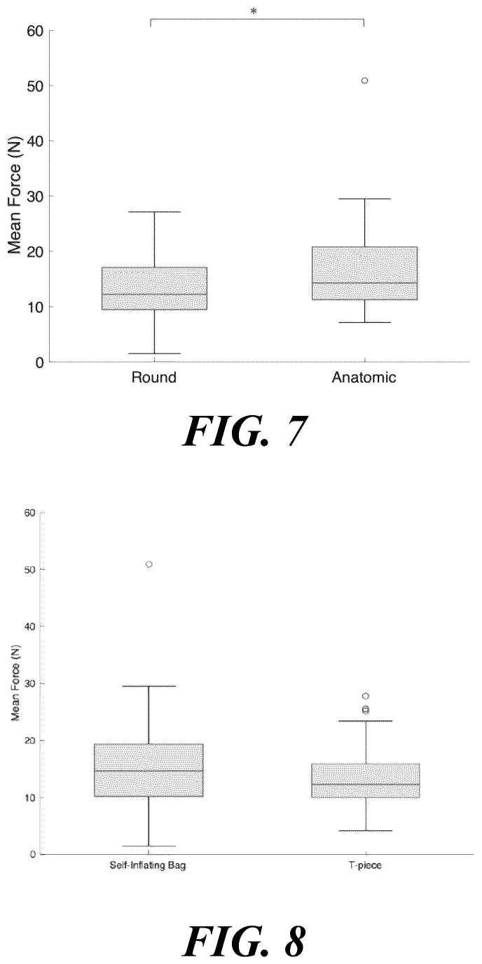

FIGS. 6-8 illustrate other factors that may be used to adjust thresholds for the system 10. For example, FIG. 6 shows the different results received for each of the different locations for the plurality of face sensors 22. FIG. 7 illustrates differences in round vs. anatomic face masks 14, and FIG. 8 illustrates differences between self-inflating bags vs. T-piece style PPV devices 12. It was uncovered that users applied less force under the head using the round compared with anatomic mask 14 (mean difference: 3.23 N, 95% CI: [1.14, 5.31 N], p=0.003), whereas a more negligible differential was uncovered comparing self-inflating bags with the T-piece (mean difference: 1.84 N, 95% CI: [−0.01, 3.69 N], p=0.052). In some embodiments, the applied forces measured via sensors 22 and/or the overall force measured via under-head sensor 32 are correlated to mask leak, peak inflation pressure, and tidal volume. Other mathematical modeling or relationships can be determined or analyzed relating to the applied forces, overall forces, mask leak, peak inflation pressure, and/or tidal volume. Also, other aspects to inform the training process may be incorporated with the GUI 46, such as monitoring chest rise, health vital signs, amount of air received, and/or status of condition. In a preferred implementation, information from breathing rate and leakage from the mask are incorporated in the feedback.

In some embodiments, training sessions combine a lecture and hands on case-based simulation, some specifically use training videos to aid the clinician. The training videos are used as a tool to increase the effectiveness of training sessions, and are stated to increase the effectiveness of the sessions by 10%. The videos address the issues of applying too much pressure, but in vague terms. The issues of applying too little pressure are addressed in the sessions in terms of preventing leakages. Another current study in the training strategies show the training sessions decreasing the leakages by 60%, but seeing an increase of 5% three weeks later. The study identified that training in mask ventilation was focused on preventing mask leakages, and not airway obstructions that are caused by applying too much force. The focus of the training sessions, as well as the limited effectiveness, demonstrates areas of improvement that are needed in the way training sessions are conducted. The focus of the present disclosure is at least partially to help improve these training sessions.

A study was conducted to characterize applied force on the face and head during simulated mask ventilation with varying mask, device, and expertise level. Participants included neonatal healthcare providers, categorized as novices and experts in positive pressure ventilation (PPV). PPV was performed for 2 minutes each in a 2×2 within-subjects design with two masks (round and anatomic) and two ventilation devices (T-piece and self-inflating bag). Applied force (Newtons (N)) measured under the head and at four locations on the manikin's face (nasal bridge, mentum, left and right zygomatic arches), and symmetry of force applied around the mask rim. For the 51 participants, force applied to the head was greater with the self-inflating bag (SIB) than the T-piece (mean (SD): 16.03 (6.96) N vs. 14.31 (5.16) N), and greater with the anatomic mask than the round mask (mean (SD): 16.07 (6.80) N vs. 14.26 (5.35) N). Under-head force decreased over the duration of PPV for all conditions. Force measured on the face was greatest at the left zygomatic arch (median (IQR): 0.97 (0.70-1.43) N) and least at the mentum (median (IQR): 0.44 (0.28-0.61) N). Overall, experts applied more equal force around the mask rim compared to novices (median (IQR): 0.46 (0.26-0.79) N vs. 0.65 (0.24-1.18) N, p<0.001). These may be used in at least some embodiments as one or more applied force thresholds.

For the study, four pre-calibrated ultrathin microforce sensors (SINGLETACT, PPS UK Limited, Glasgow, UK) were applied on the manikin's face at the nasal bridge, mentum, and the left and right zygomatic arches. Force (Newtons (N); 1 N=102 gram-force) under the head was measured with a modified load cell (SPARKFUN ELECTRONICS, Niwot, CO, USA) concealed from view with a blanket. Forces on the face and under-head were continuously measured at 20 Hz using a microcontroller and custom data collection script. Initial values from each force sensor at resting state were recorded for each trial and were subtracted from the time series such that all sensors had a zero datum when unloaded. A wireless respiratory function monitor (MONIVENT, Gothenburg, Sweden) was placed between the mask and the PPV device.

A linear mixed effects (LME) model was fit to characterize the forces measured under the manikin's head for each condition over time. A repeated-measures analysis of variance (ANOVA) was fit to characterize the relationship of mask type, device type, experience level, and force sensor location on the force measured at the face-mask interface. An additional metric, asymmetry index, was calculated as the square root of the sum of squares of the face force sensor differences. The asymmetry index describes how evenly force is distributed around the mask rim, with zero indicating perfectly equal force distribution in one embodiment. A second repeated-measures ANOVA was fit to characterize the relationship of mask type, device type, and experience level on the asymmetry index. A Pearson correlation was used to assess linearity between the under-head force and asymmetry index. Data were presented as mean (SD) for normal distributions and median (IQR) for skewed distributions. Dependent t-tests were used to compare results from within-subjects factors, mask and device type, and independent t-tests were used to compare result for the between-subjects factor, expertise. Statistical analyses were performed with MATLAB 2022 edition (MATHWORKS, Natick, MA, USA).

Participants were categorized into novices and experts. Under-head force varied by mask and device type. Forces were greater when using the SIB compared to the T-piece resuscitator (mean (SD): 16.03 (6.96) N vs. 14.31 (5.16) N, p=0.002), and greater when using the anatomic mask compared to the round mask (mean (SD): 16.07 (6.80) N vs. 14.26 (5.35) N, p=0.005). The greatest mean under-head force was observed for the SIB with anatomic mask (17.30 (7.96) N). Experts and novices applied the least mean force with the T-piece with round mask (12.99 (4.47) N and 14.65 (5.76) N, respectively). The LME model illustrates that, on average, under-head force decreases over the duration of each PPV trial (−0.032 N/s, p<0.0001) for all conditions, with small differences in slope depending upon condition and expertise. This primary effect is equivalent to a 3.84 N decrease in under-head force over the two-minute trial, which may be used as an applied force threshold in accordance with one or more embodiments.

A logarithmic transformation was applied to face force sensor data prior to statistical analysis. Mask type and device type had no effect on force measured at each face sensor location. However, forces differed depending upon HCP experience level and location of the sensor on the manikin's face. Although novices applied a greater force across all sensors on the face compared with experts (median (IQR): 0.75 (0.48-1.09) N vs. 0.74 (0.48-1.06) N, p<0.001), the effect was negligible (Cohen's d-0.05). The greatest force was applied at the left zygomatic arch (median (IQR): 0.97 (0.70-1.43) N) and the least force was applied at the mentum (median (IQR): 0.44 (0.25-0.61) N). When comparing handedness to forces applied at each face sensor location, a relationship emerged that corresponds to higher force on the side of the mask-holding hand. Individuals who held the mask with their right hand (n=8) had larger applied force at the right zygomatic arch than individuals who held the mask with their left hand (median (IQR): 1.13 (0.75-1.50) N vs. 0.86 (0.68-1.13) N, p=0.028). The ANOVA model did not support an effect of mask or device type on the asymmetry index, which may be used as a factor in the analysis. However, novices had a significantly higher asymmetry index than experts (median (IQR): 0.65 (0.24-1.18) N vs. 0.46 (0.26-0.79) N, p<0.001), indicating experts applied force more symmetrically.

The objectives of the study were to characterize applied forces on the face and head during simulated PPV and assess the effects of mask type, device type, and experience on applied forces. It was found that mean under-head force was higher when using the SIB and anatomic mask and applied force decreased over the time of the trial, which may be used to guide the parameters for the ventilation assessment system 10 and methods. In addition, it was found that forces measured on the manikin's face were applied asymmetrically, with the greatest force applied to the zygomatic arch on the operator's hand-holding side and the lowest force applied to the mentum. Asymmetry was related to experience, as novice resuscitators applied more asymmetric force compared with expert resuscitators.

Controlling for mask type and experience, mean under-head force was lower when clinicians used the T-piece compared to the SIB. This force decrease may be attributed to the more strenuous posture required when using the SIB. The vertical alignment of the T-piece with the mask, neutral posture, and smaller motions required to use a T-piece could result in lower force applied to the manikin's head.

For further context, the applied force was converted to pressure (mmHg), by dividing the measured force (N) by the sensor surface area (face sensors: 5.03 cm2, occiput contact area with under-head sensor: 15.90 cm2) and the pressure was compared to the commonly referenced threshold (32 mmHg) for preventing soft tissue injury in adults and older children. We found that the pressures measured on the manikin's face were much smaller than this threshold (6.56-14.46 mmHg) while under-head pressure exerted with either device (67.27-81.61 mmHg) exceeded it. Given the lower mean arterial pressure in newborns, the threshold likely underestimates the potential for soft tissue injury in the neonatal population. This raises concern that if the under-head pressures exerted during training sessions with a manikin were sustained during actual resuscitations, they could lead to injury. This potential for injury emphasizes the need to ensure that the fidelity of training devices, where learners acquire and practice skills, reflects the actual clinical environment. If technical features including the quality of the simulated skin where it contacts the mask, the structure of the manikin's airway, and the compliance of simulated tissues are not realistic, learners may develop dangerous habits in the training environment that are translated to the clinical environment. Furthermore, these findings support the recommendation to use a soft mattress below the newborn's head during PPV to distribute forces and minimize the risk for tissue injury.

The results showed that clinicians applied less force when using the round mask compared to the anatomic mask. This reduction in under-head force could arise from the mask design or holding method. The round mask uses a thin, membrane rim to create a seal while the anatomic mask uses an inflated rim. Participants held the round mask by grasping the stem and applying force to the connector positioned between the PPV device and mask. In contrast, the one hand C-E hold was used with the anatomic mask, requiring the operator to apply force directly to the rim of the face mask. This technique causes increased surface contact between the mask-holding hand and the manikin's face. The reduced force with the round face mask raises the question if there was enough force to secure the mask to the manikin's face, avoid leak, and deliver sufficient tidal volume. In some implementations, peak inspiratory pressure and leak around the mask can be measured as well to help achieve effective ventilation.

Average under-head force decreased over the duration of PPV across all conditions, though the magnitude varied slightly depending on condition and expertise. This outcome could be a result of accumulating fatigue over the 4 two-minute trial periods. Alternatively, participants may have applied a large force when beginning ventilation and decreased the force once they perceived they were achieving the desired peak pressure and chest movement. Measuring clinician muscular activity, applied forces, fatigue, and ventilation metrics over time during simulated PPV in future studies could provide additional data to address these hypotheses.

When comparing forces at each face sensor 22 location, it was observed that force was unevenly applied around the mask rim 52. The greatest forces were observed at the zygomatic arch corresponding to the mask holding hand. In addition, novices applied more asymmetric force around the mask rim than experts. The lack of correlation between under-head force and the asymmetry index indicates that asymmetric force distribution on the face is not a direct result of insufficient or excessive force applied to the head and may occur at both low and high applied forces. These findings emphasize the importance of learning good mask placement during PPV training. Real-time feedback showing trainees how forces are applied on the manikin's face during training sessions could be a useful adjunct to improve the acquisition of PPV skills.

The use of a manikin may introduce some limitations in the ability to translate results to newborn infants. The mechanical properties of the manikin's head and skin may differ from an infant, which could result in discrepancies between the exact measurements reported here and the applied force in a clinical setting. Accordingly, adjustments can be made whether this is being used on an infant patient as opposed to a manikin. In addition, as opposed to four discrete sensors 22 located around the rim 52, measuring all contact points may be desirable in other embodiments, including forces applied to lift the jaw, as they could provide additional information about mask placement and ventilation strategy.

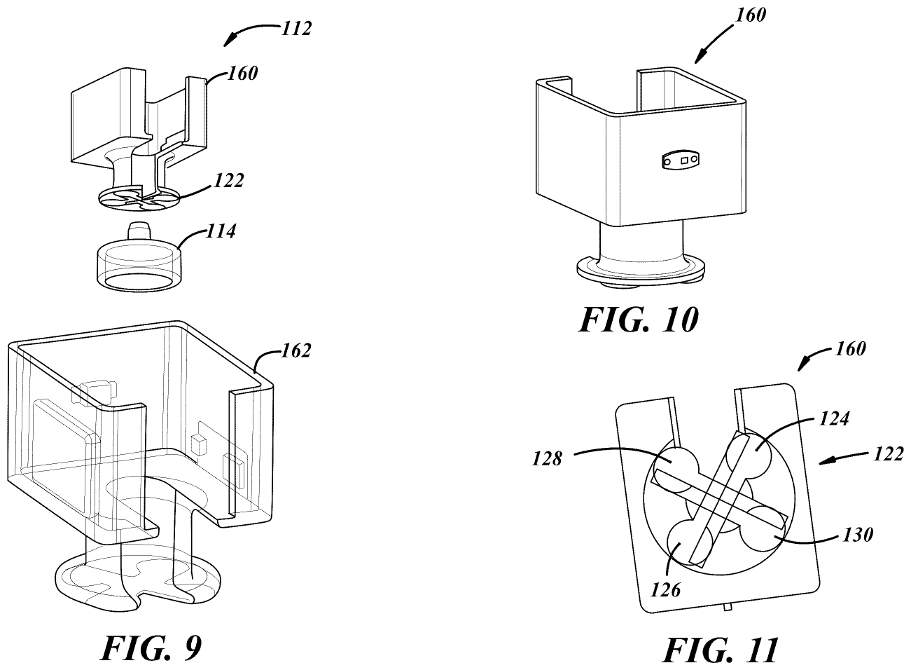

FIGS. 9-11 show another embodiment of a PPV device 112 that could be used with the ventilation system 10 (like reference numerals in the figures denoting like components). In this embodiment a sensor adapter 160 is included which holds the sensors 122, with the sensor adapter being located in a housing 162. In this embodiment, the device 112 is a 3D printed object that is placed around a round neonatal mask 114 and T-piece during PPV. There are sensors 122 located at the bottom of the object, which measure the force applied to the top of the mask 114. The device 112 may also include a housing 162 for the electrical components. The device 112 had success with portability, durability, and cost effectiveness. However, the material PLA was rigid, and accordingly, a more pliable material for the sensor adapter 160 could be employed to help improve consistency and accuracy.

Relevant standards that may be applicable when designing the ventilation system 10 and methods herein include, but are not limited to, ISO 14971, 10992, 62304, 13485, and IEC 60601. For the device 12, specifications such as weight, ability to measure applied force, and the ability to provide live feedback were of particular importance, with other factors such as size, set up time, and training, to cite a few examples, also being at least partially considered. Table 1 includes example specifications that may be considered:

TABLE 1

| Category | Requirement | Specifications |
|---|---|---|
| Functionality | Allows user to | One-handed CE hold, two-handed hold, |
| | perform four hand | spider hold, and stem hold |
| | positions | |
| | Does not contribute | <25% air leakage (Low Leak - minimal) |
| | to excessive air | <10% air leakage (Optimal) |
| | Accurately detects | Force sensing range: 0 -10N |
| | forces in the | Resolution: < 0.045N |
| | correct range | |
| | Measures force in | Nasal bridge, left and right zygomatic arch, |
| | four key locations | and mentum |
| | Outputs readings | Vary <5% (considering a factor of |
| | proportional to | proportionality) |
| | readings from prior | |
| | testing | |
| | Supports the most | 60 mm round mask (minimal) |
| | common training | 35-60 mm round mask (optimal) |
| | masks | |
| | Provides real time | ≥1 sensory feedback signal |
| | feedback for the | <250 ms of reaching target force range |
| | user | ≥4 locations |
| Practicality | Is transferable | No residue |
| | | Separable from manikin |
| | | ≤2 tools required to transfer |
| | Is safe to use | No to minimal risk of shock |
| | | No to minimal sharp edges or surfaces |
| | Is durable | Withstands >1250 10-minute cycles Withstands |
| | | 40N applied force |
| | Is lightweight | <600 grams (minimal) |
| | | <200% of weight of the respirator (optimal) |
| | Can be set up | <1 minute for complete set up (hardware and |
| | quickly | software) |
| | Is easy to use | Device takes <25 cycles to learn how to use |

While multiple techniques are used to hold the face mask, four commonly recognized hand positions are one-handed CE hold, two-handed hold, spider hold, and stem hold. For the device 12 to accurately simulate neonatal resuscitation correctly, it may be desirable for all of these hand holds to be achievable while using the training device 12. The divergence in hand holding techniques implies that each four of these hand holding positions should be obtainable with the device 12 being applied, and therefore, the device 12 is unlikely to interfere with the natural holding positions.

In some embodiments, when air leakage is assessed, a low leak limit may be used as a threshold for the GUI 46. In one embodiment, Low Leak occurs when less than 33.3% of the air supplied escapes. Noticeable air leakage can be a form of feedback for nurses as they try to avoid it; this can lead them to press harder to form a better seal between the mask-face interface. To be a useful training device, minimizing excessive air leakage is desirable to properly emulate the real scenario so that nurses do not learn to press harder than necessary.

It may be desirable to utilize sensors 22 that can measure the appropriate magnitude of forces that are typically applied to the neonate's face during the PPV process. Detecting the correct forces can help avoid unreliable and insufficient data collection. In one example, the applied force is about 0-10 N. The resolution of the sensor readings is preferably less than 0.045 N. In this implementation, the resolution number is derived from the Weber Weight Discrimination Experiments, which determined that the minimum difference in force a human can detect while pressing on an object correlates to a factor of 0.09 in relation to the force being applied. Since the minimum force applied to the mask is 0.5 N in one embodiment, the corresponding minimum detectable force would be 0.045 N.

In some embodiments, such as those illustrated in FIGS. 9-13, a sensor adapter 160, 260 is used to hold the sensors 122, 222 such that they can be used on different mask styles. In advantageous implementations, the sensor adapter 160, 260 can be used along with a full range of 35-60 mm neonatal round or anatomical masks.

As detailed herein, the system 10 is configured to provide real-time feedback. “Real-time” may not be immediate as used herein. For example, in one embodiment, the response time was set to be 250 ms (i.e., “real-time”), this being the response time to sensory input of humans. This was compared to specifications for similar medical devices and was comparable to achieving actual real-time feedback. This real-time feedback may occur separately for each of the sensors 22 to help ensure that an equal load is being applied.

In some implementations, the device 12 is transferable, meaning that the training device is not a permanent fixture and is separable from the manikin 38. With such an implementation, the device 12 should not leave a physical trace such as residue on the manikin 38 after its use. Lastly, to easily integrate the device, it should require ≤2 tools to transfer. This transferability and integration can help with widespread adoption in hospitals, maximizing its accessibility and impact.

Durability of the device 12 may also be considered. The specifications for the durability of the device 12 include that it should be able to withstand at least 1250 ten-minute cycles and can survive an applied force of 40 N. The 1250 cycles comes from an estimation based on the following parameters: expected shelf life, number of uses per training, number of trainees per session, number of devices per hospital, and number of training sessions held per year. The expected shelf life of healthcare respirators and masks is five years, so the same approximate lifetime is expected here. Since typical mask ventilation training requires a median of 25 procedures, usage rate of about 25 times per trainee in each training session can be assumed. For estimation purposes, it was assumed that there would be 2 PPV training sessions with five new trainees per hospital held annually and one device provided per hospital. Taking these parameters into consideration, it was estimated that the device 12 needs to sustain 1250 cycles. The 10 minute cycle length is from the maximum time required to complete a PPV procedure. The device 12 should also be able to survive a 40 N applied force to ensure that it can handle the maximum force applied during PPV, which is about 10 N in one embodiment. Other considerations include, but are not limited to, the device 12 being lightweight (e.g., about 100 g), capable of being set up quickly, and being generally easy and safe to use.

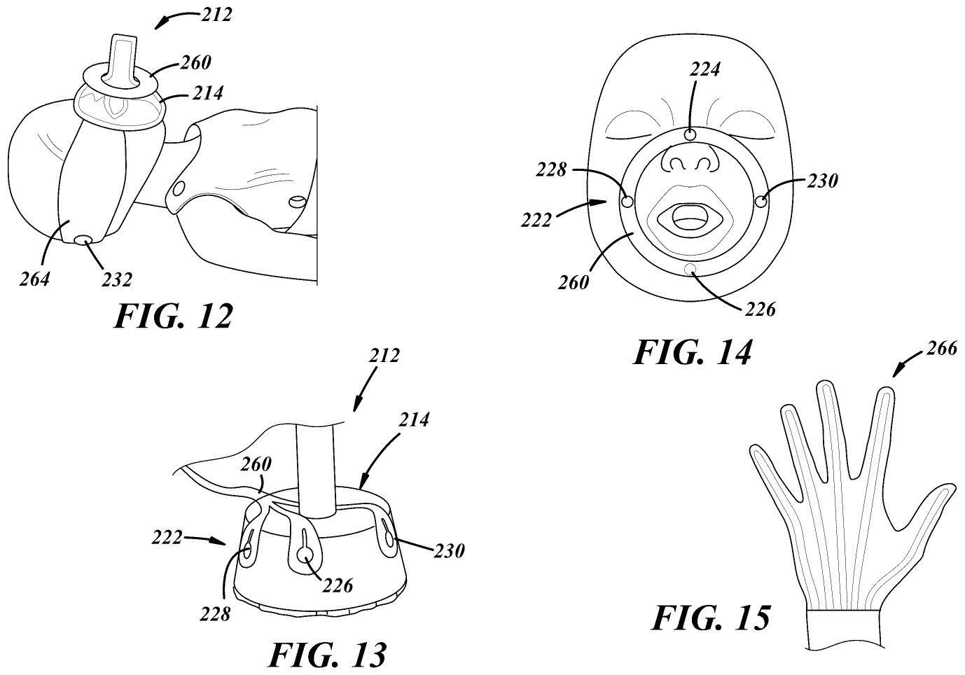

FIGS. 12-14 show an embodiment 212 where a sensor adapter 260 integrates the sensors with a body that can be fitted over the mask 214 (FIGS. 12-13), or under the mask (FIG. 14). Additionally, with the embodiment shown in FIG. 12, a strap 264 is included to positionally locate the under-head sensor 232. This arrangement can provide for a more integrated device 212 implementation. The sensor 232 can measure the amount of force being applied to the bottom of the manikin head as the strap 264 holds the sensor 232 (and potentially other sensors) in place. Additionally, FIG. 15 shows a sensing glove 266 that may include one or more capacitive sensors or other sensors that may be used with the system 10. The sensing glove 266 includes embedded sensors and can be worn by the clinician performing PPV. The glove 266 would then measure the amount of force being applied to the manikin's face and could provide live haptic feedback. In various embodiments, feedback may include visual, audio, haptic, or some combination or sub-combination thereof.

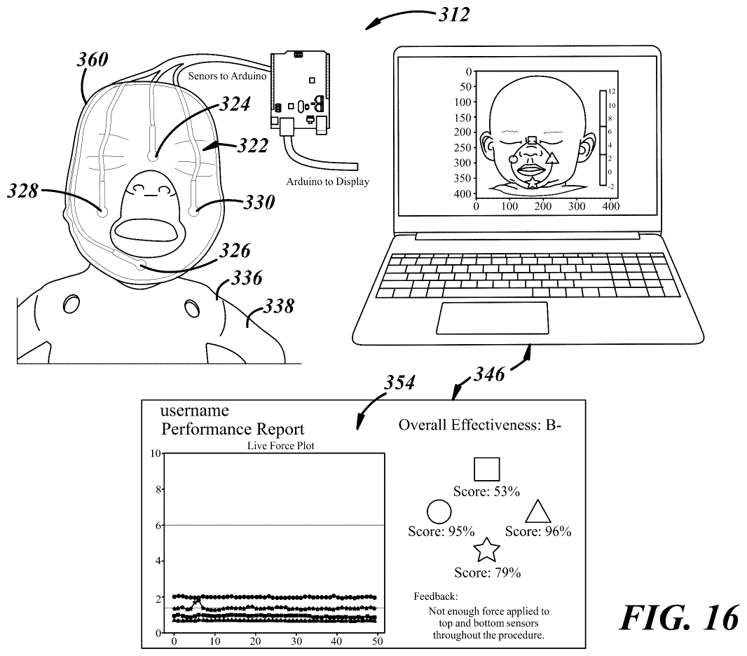

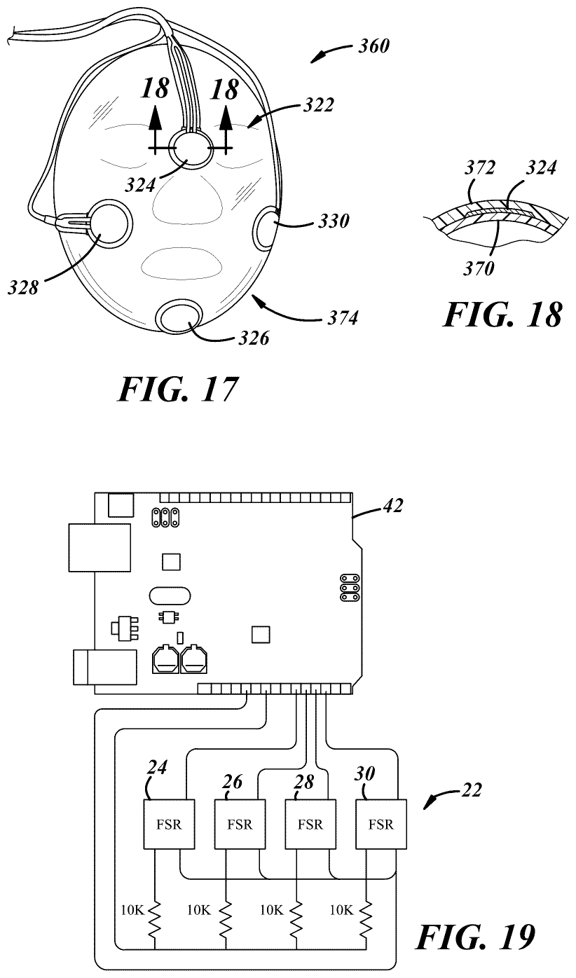

FIGS. 16-18 illustrate another embodiment of a PPV device 312, and more particularly, a sensor adapter 360 that includes the plurality of sensors 322 and at least some of the wiring sandwiched between two film layers 370, 372. In some implementations, the sensor adapter 360 is the face mask itself. The film layers 370, 372 are conformational interlayers that are configured to be situated between the manikin 338 and mask (not shown). In this embodiment, as shown in FIG. 18, the sensor 324 is sandwiched between two film layers 370, 372, but more or less layers could be employed. The thickness of each layer 370, 372 is about 1-4 mm, with closer to one being preferred so as to have an overall mask thickness that is about 2 mm or less. In the illustrated implementation, the layers 370, 372 are vacuum formed or otherwise contoured to at least partially conform to the face of the manikin 338. Accordingly, the sensor adapter 360 has a face contour profile 374 that is advantageously flexible and provides a more conformal arrangement for the sensors 322. The face contour profile 374 has a mouth cutout that exposes the nostrils of the manikin 338.

With the GUI 346, the information can be colored or otherwise visually altered to help with readability. To indicate the appropriate force range, the scale can be color-coded into three distinct categories: red for excessive force, green for the correct force, and purple for insufficient force. To accommodate individuals with color vision deficiencies, system settings can be implemented that adapt the colors according to the type of color blindness of the user, mirroring the functionality of other computer programs. Post-training, users can have access to a continuous time-series graph displayed on a summary page of the GUI 346, which will provide a detailed visual representation of the force exerted at each of the key locations over the duration of the training session.

In operation, generally, the first step of the procedure is to place the adapter 360 on the manikin 338, with the wires already attached to the sensors 322 within the adapter and grouped together in order to not interfere with the training space. Next, with the wires already connected to the processor, the processor then connects to the display device or GUI 346, in this case being a computer with code through Python. Next, the user places a mask and begins the resuscitation process as usual. The computer will then display the force results at each of the four individual pressure sensors with the associated color indicating if they are in the right range of force, or too high or too low. The location of the display device can be straight ahead, eye level from the user in order to maximize the efficiency of reading the results. The user can then adjust the force applied accordingly.

In the device 312 implementation. the material used for adapter 360 was elastic in order to fit to the shape of the manikin's face as it deforms during typical PPV application. The elastic material may perform better than hard plastic polymers that are not compatible with thermoforming. The primary candidate for the adapter 360 is a thermoplastic urethane material (TPU). TPU is commonly used in thermoforming and molding applications, and is known for its elastic properties and durability. TPU also is lower cost than other materials and fits with applicable safety requirements. TPU is considered to be biocompatible for medical devices and is considered a safer alternative to other plastic materials. Other example materials for a sensor adapter may include a thermoplastic elastomer (TPE) or rubber, to cite a few non-limiting examples. Due to the innovative and efficient manufacturing method of thermoforming to the face contour profile 374 of the manikin 338 and embedding the sensors 322 between two layers 370, 372, the design is scalable to different manikin shapes and sizes. Minimizing spacing between the adapter 360 and the manikin's face is generally desirable.

With the adapter 360, after testing, it was discovered that there is a clear correlation between the thickness of the TPU layers 370, 372 and the force readings from the force sensors 322. As layers of TPU are added in between the sensor and the mass, the magnitude of the force readings become increasingly damped, especially at low force magnitudes. The low end of the range of measurable force is also truncated when adding TPU layers. The smallest detectable measurement that was achieved for 1 TPU layer is 120 grams (˜1 N), and the smallest detectable measurement for two TPU layers is 170 grams (˜1.5 N). With no layers of TPU, the sensors were able to distinguish weights as low as 70 grams (˜0.7 N). It is also important to notice that at low force magnitudes, the data appeared to fit a curved polynomial trendline, meaning that the resistance of the force sensor at low forces (<500 g) is very sensitive. Due to this high sensitivity and quality of the sensors, large error bars were seen on the low end of the force measurements. At larger weights (>500 g), the sensor readings follow a linear trend and are much more consistent. The inconsistency at low weights as well as the truncated measurement range could potentially be mitigated by using more expensive, high-quality force sensors, such as the SINGLETACT sensor. Additionally, it is possible to mathematically calibrate the device 312 to accommodate the damping effects of the TPU.

Other features of this embodiment includes soldered wiring that connects at a tail toward the top of the head of the manikin 338. This can help during manufacture or the lamination process between layers 370, 372, and it aims to mitigate potential failure points associated with repeated pulling, helping to ensure longevity and durability of the design.

In some embodiments, with respect to the GUI 46, 346, a post-training summary page can be included that features a comprehensive performance report, providing users with valuable insights into their training session. This report can include a continuous time graph displaying performance data throughout the session, metrics on the four key locations, and a performance grade to inform users of their training session efficacy. Furthermore, user performance data can be saved and tracked over multiple sessions, allowing users to monitor their progress and track improvements over time.

FIG. 19 shows an example schematic that can be used to connect the processor 42 with the sensors 22. As mentioned, the processor 42 in this embodiment is an ARDUINO, which may be an ARDUINO NANO or an ARDUINO UNO R3 to cite a few examples, but other processor platforms are certainly possible. Additionally, other sensor types are possible, such as a round force-sensitive resistor, FLEXIFORCE sensors, or a flexible thing film ring pressure sensor, to cite a few examples.

It is to be understood that the foregoing description is of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all the following: “A”; “B”; “C”; “A and B”; “A and C”; “B and C”; and “A, B, and C.”

1. A method of assessing ventilation, comprising the steps of:
receiving a measurement of an applied force between a face mask and a face of a patient from a first sensor located at least partially between the face mask and the face of the patient;
receiving a measurement of an overall force applied to the patient from a second sensor; and
contextualizing the measured applied force and the measured overall force to provide a graphical user interface.

2. The method of claim 1, wherein the graphical user interface includes a time series plot of an output from each of the first sensor and the second sensor.

3. The method of claim 2, further comprising the step of accounting for an initial unloaded value of each of the first sensor and the second sensor in the time series plot.

4. The method of claim 1, wherein the measurement from the first sensor, the measurement from the second sensor, or the measurements from both of the first sensor and the second sensor are used to provide an indication of a potential airway obstruction.

5. The method of claim 1, wherein the first sensor is one of a plurality of face mask sensors.

6. The method of claim 5, further comprising the steps of comparing each sensor measurement of the plurality of face mask sensors to assess similarity of the applied force across the face mask.

7. The method of claim 6, wherein an increased relative pressure at one force sensor in the plurality of face mask sensors is used to provide an indication of a potential injury.

8. The method of claim 5, wherein the second sensor is an under-head sensor configured to be located on an opposite side of a head of the patient from the face.

9. The method of claim 1, further comprising the step of determining a relationship of the applied force and/or the overall force to one or more of a mask leak measurement, a peak inflation pressure, and a tidal volume.

10. The method of claim 1, further comprising the step of adjusting an analysis depending on a one-hand usage pattern or a two-hand usage pattern.

11. The method of claim 1, further comprising the step of calculating a force asymmetry index for the graphical user interface.

12. A non-transitory, computer-readable storage medium storing instructions thereon that when executed by one or more electronic processors causes the one or more electronic processors to carry out the method of claim 1.

13. A ventilation assessment system, comprising:
a plurality of face mask sensors configured to be situated between a face mask and a face of a patient to measure an applied force between the face mask and the face of the patient; and
an under-head sensor configured to measure an overall force applied to the patient.

14. The ventilation assessment system of claim 13, wherein the plurality of face mask sensors are attached to or integrated into the face mask.

15. The ventilation assessment system of claim 13, wherein the patient is an infant manikin and the plurality of face mask sensors are integrated into or onto the face of the infant manikin.

16. The ventilation assessment system of claim 15, wherein the under-head sensor is integrated into or onto a head of the infant manikin.

17. The ventilation assessment system of claim 15, wherein the under-head sensor is integrated onto the head of the infant manikin with a strap.

18. The ventilation assessment system of claim 13, wherein the plurality of face mask sensors is integrated with a conformational interlayer.

19. The ventilation assessment system of claim 18, wherein the conformational interlayer includes two or more thermoplastic layers that have a face contour profile, and the plurality of sensors are sandwiched between the two or more thermoplastic layers.

20. A face mask for a ventilation assessment system, comprising:
a nasal bridge sensor configured to measure an applied force across a nasal bridge of a patient; and
a mentum sensor configured to measure an applied force at a mentum of the patient, wherein the nasal bridge sensor and the mentum sensor are at least partially aligned with and at least partially overlapping a longitudinal axis of the face mask.

21. The face mask of claim 20, comprising a right zygomatic arch sensor configured to measure an applied force at a right zygomatic arch of the patient and a left zygomatic arch sensor configured to measure an applied force at a left zygomatic arch of the patient.
